(19) United States
(12) Patent Application Publication
(10) Pub. No.: US 2025/0001147 A1
(43) Pub. Date: Jan. 2, 2025

(54) Cylindrical Microneedle Wireless Capsule System and an Operation Method Thereof

(71) Applicant: National Chin-Yi University of Technology, Taichung City (TW)

(72) Inventor: Jian-Xing WU, Taichung City (TW)

(21) Appl. No.: 18/513,989

(22) Filed: Nov. 20, 2023

(30) Foreign Application Priority Data
Jun. 29, 2023 (TW) 112124399

(51) Int. Cl. A61M 31/00; A61M 37/00

(52) U.S. Cl. CPC A61M 31/002; A61M 37/0015

(57) ABSTRACT

The invention relates to a cylindrical microneedle wireless capsule system and an operation method thereof, comprising a cylindrical microneedle wireless capsule and a control component of the cylindrical microneedle wireless capsule thereof. The functionality of the cylindrical microneedle wireless capsules of the present invention is used for an oral delivery, and for serving as a platform for a variety of biopharmaceutical therapeutic drug molecules that are currently limited to injection.

### BACKGROUND OF THE INVENTION

### 1. Field of the Invention

The invention is related to a capsule system and an operation method thereof, and more particularly, to a cylindrical microneedle wireless capsule system and an operation method thereof.

### 2. Description of the Prior Art

As “biopharmaceuticals” are particularly sensitive to human protease, endonuclease, bacteria, or the extreme pH values, present in the human gastrointestinal tract, when “biopharmaceuticals” are introduced into the human body by orally delivering up, which could be undermined by the abovementioned human protease, endonuclease, bacteria, and extreme pH values, resulting in the loss of the drug effectiveness, therefore, “biopharmaceuticals” only can be delivered effectively through injection to achieve therapeutic effects.

The delivery mechanism of the abovementioned “biopharmaceuticals”, as evident from numerous recent research literature, is predominantly supported by using “needling” methods to bypass the gastrointestinal barrier. This “needling” approach is highly effective and safe. For instance, diabetes patients require daily insulin injections, and even with every meal and before sleeping, these diabetes patients must self-administer insulin injections. The pain caused by these injections is practically daily and lifelong for these diabetes patients. If an injection is delayed, cardiovascular diseases may develop, and clinical statistics show that once patients suffer due to poor blood sugar control, they may lose an average of 12 years of life.

Moreover, the current medical capsule technology for delivering “biopharmaceuticals”, despite years of development, has the following drawbacks in medical practice:

(1) Specific locations at specific times cannot be monitored,

(2) Movement only rely on gastrointestinal peristalsis,

(3) the medical capsule technology system can't be controlled for actions like acceleration, stopping, and reversing, and

(4) most experimental endoscopic capsules are too large for the size, with module reaction speeds that are particularly slow, making the efficiency and agility low and unsuitable for more complex treatments.

As previously mentioned, “biopharmaceuticals”, as defined by the “Regulations for Registration of Medicinal Products” in our country, are “serum, antitoxin, vaccines, toxoids, and bacteria sap, etc., produced based on the principles of microbiology and immunology”. In other words, “biopharmaceuticals” include toxins, toxoids, allergens, vaccines, genetically engineered products, blood-derived products, cell therapy products, gene therapy, and other large molecule drugs. “Biopharmaceuticals” frequently appear in daily life, such as insulin for treating diabetes, antivenom for snake bites, flu vaccines taken in autumn and winter, chickenpox vaccines for children, and the human papillomavirus vaccine for preventing cervical cancer, all of which belong to “biopharmaceuticals”.

### SUMMARY OF THE INVENTION

The present invention is a cylindrical microneedle wireless capsule system and an operation method thereof. The system comprises a cylindrical microneedle wireless capsule and a control component, wherein, the control component is electrically connected to the cylindrical microneedle wireless capsule, forming the cylindrical microneedle wireless capsule system together.

The cylindrical microneedle wireless capsule of the invention mainly consists of a flexible plastic outer shell hinge; a ring-shaped magnet and miniature motor controller capable of transporting biopharmaceuticals (insulin); a biopharmaceutical (insulin) drug chamber; a magnetic sensor; and a microneedle. The microneedle is installed inside the biopharmaceutical (insulin) drug chamber, the ring-shaped magnet and miniature motor controller are positioned above the drug chamber, the flexible plastic outer shell hinge is positioned above the ring-shaped magnet and the miniature motor controller, and the magnetic sensor is located above the flexible plastic outer shell hinge.

The control component of the cylindrical microneedle wireless capsule in the invention comprises a dedicated integrated circuit, a field-programmable gate array controller, a wireless antenna module, a power light, and a battery.

The control component of the cylindrical microneedle wireless capsule includes the dedicated integrated circuit which contains a power decoder, an injection control structure array, a high voltage charge pump, a pulse counting circuit, a column decoder, and a data serializer.

The operation method of the cylindrical microneedle wireless capsule system of the invention starts with the “beginning” step, followed by the “moving to the desired injection position” step, then the “determining if an electrical stimulation position” step, followed by the “waiting for the remote end to send/receive instructions” step, then the “generating the first electrical pulse to set a timer for counting” step, next is the “generating a second electrical pulse to set a timer for counting” step, followed by the “determining whether to continue stimulation or not” step, then the “reducing the electrical pulse timer count” step, subsequently the “determining if the electrical pulse timer count equals to zero” step, and finally, the “ending” step.

The advantages of the cylindrical microneedle wireless capsule of the present invention include the high compatibility and the ability to be remotely controlled to manage the microneedles.

The advantages of the cylindrical microneedle wireless capsule of the present invention include a biopharmaceutical-release magnetic capsule endoscopy device structure which is easy to swallow.

The advantages of the cylindrical microneedle wireless capsule of the present invention include the magnetic capsule which can be manipulated by using an external permanent magnet, and the magnetic microneedle capsule is fixed onto the tissue wall by magnetic forces and can rotate via magnetic torque.

The advantages of the cylindrical microneedle wireless capsule of the present invention include the ability to provide linear movement behavior. The direction of the stable moving magnetic capsule is controllable, especially achieving steadiness during nonlinear motion processes.

The advantages of the cylindrical microneedle wireless capsule of the present invention include the functionality as an oral delivery aid, and serving as a supplementary platform for numerous biopharmaceutical treatments, which currently are limited to injections.

The advantages of the cylindrical microneedle wireless capsule of the present invention are the utilization of swallowable FPGA capsule technology, integrated with microneedle capsule technology, to assist in delivering biopharmaceuticals to parts of the body through the gastrointestinal tract.

The advantages of the cylindrical microneedle wireless capsule of the present invention include delivering biopharmaceuticals with the aid of the gastrointestinal tract. Besides not causing any painful sensations, the pain patients feel from daily skin injections can be reduced.

For achieving the abovementioned purposes, characteristics and advantages of the invention can be understood much more obviously, the following embodiments and the Figures are attached for detailed description as follows.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1A illustrates the cylindrical microneedle wireless capsule of the present invention.

FIG. 1B depicts the ingestion of the cylindrical microneedle wireless capsule of the present invention.

FIG. 1C shows the cylindrical microneedle wireless capsule of the present invention reaching the measurement position.

FIG. 1D illustrates the cylindrical microneedle wireless capsule of the present invention releasing biopharmaceuticals.

FIG. 1E depicts the operation method of the cylindrical microneedle wireless capsule system of the present invention.

FIGS. 2A, 2B, 2C, and 2D display the action schematic of the microneedles in the cylindrical microneedle wireless capsule 100 of the present invention.

FIG. 2A is a diagram showing the excitation/contraction of the puncture needle.

FIG. 2B depicts the ingestion of the capsule.

FIG. 2C illustrates the capsule reaching the measurement position.

FIG. 2D shows the process of drug release.

FIG. 3 depicts the control component of the cylindrical microneedle wireless capsule of the present invention.

### DESCRIPTION OF THE PREFERRED EMBODIMENT

The abovementioned and other technical contents, characteristics and performance of the invention can by present clearly in the detailed description of a preferred embodiment through cooperating with the description of the figures.

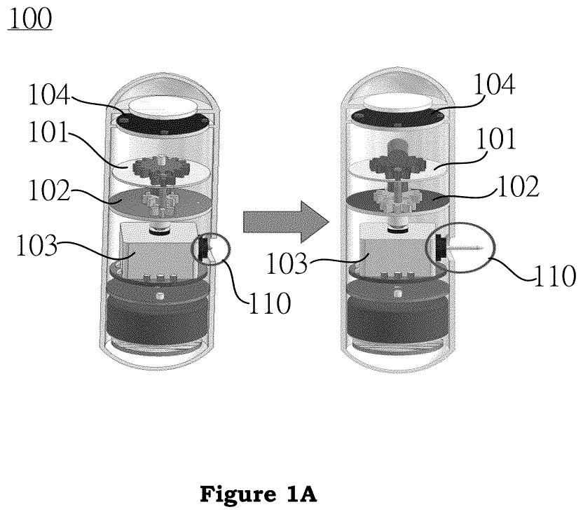

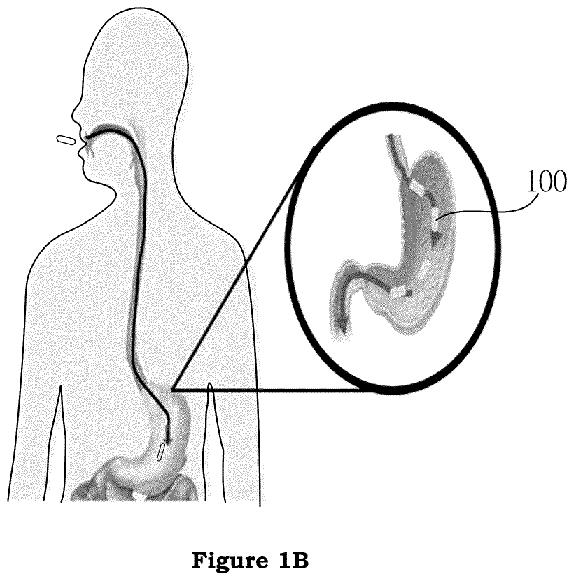

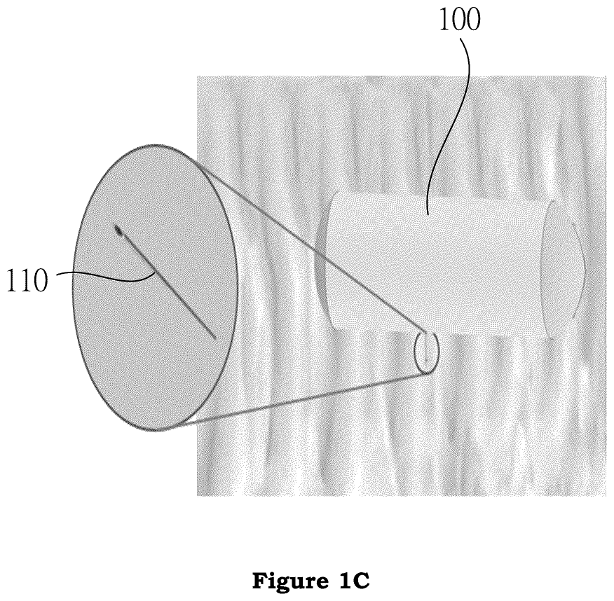

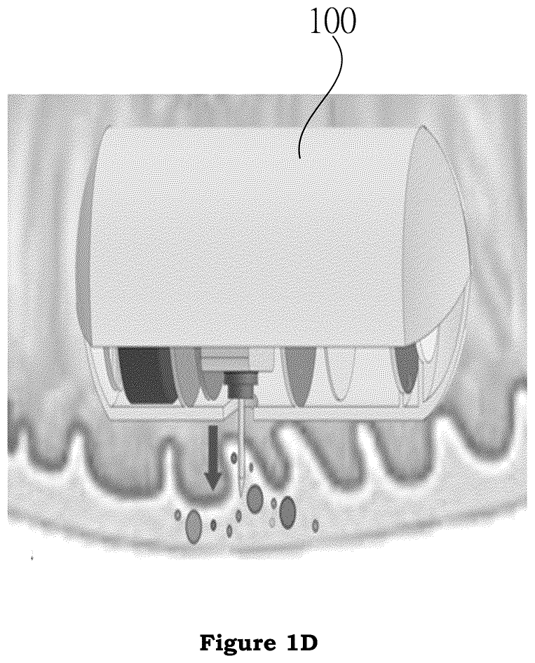

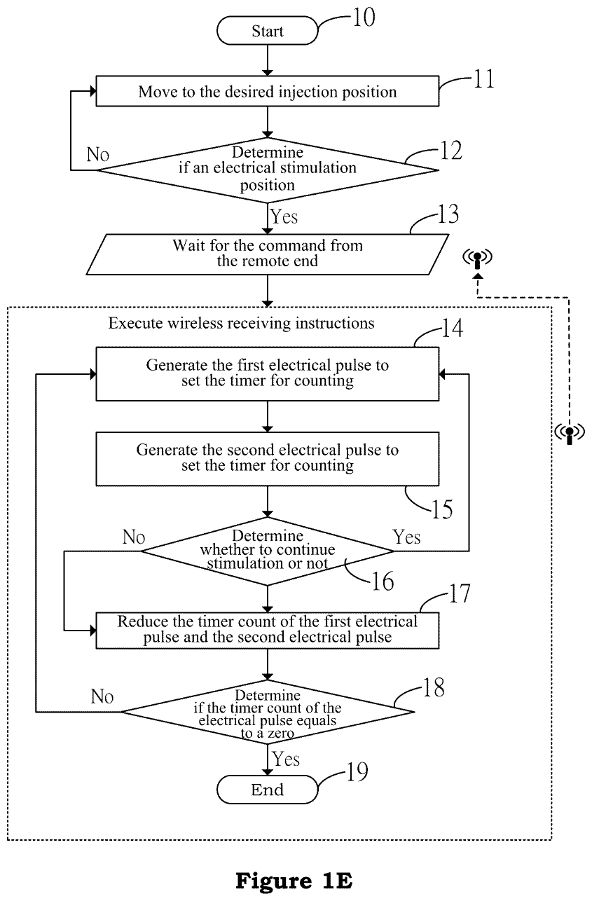

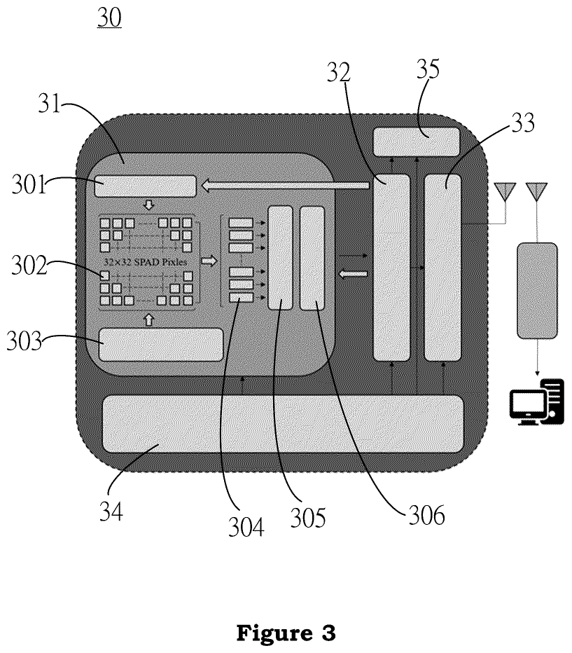

The present invention is a cylindrical microneedle wireless capsule system, comprising a cylindrical microneedle wireless capsule 100 (as shown in FIGS. 1A, 1B, 1C, and 1D), and a control component 30 of this cylindrical microneedle wireless capsule (as shown in FIG. 3). The control component 30 of the cylindrical microneedle wireless capsule electrically connects to and controls the cylindrical microneedle wireless capsule 100, together forming the cylindrical microneedle wireless capsule system.

As shown in FIG. 1A, the cylindrical microneedle wireless capsule 100 of the present invention primarily consists of several components:

A flexible plastic outer shell hinge 101.

A ring-shaped magnet and miniature motor controller 102, which owns the function to move biopharmaceuticals (insulin).

A biopharmaceutical (insulin) drug chamber 103.

A magnetic sensor 104.

Microneedles 110. These microneedles 110 are installed inside the biopharmaceutical (insulin) drug chamber 103. The ring-shaped magnet and miniature motor controller 102 are located above the biopharmaceutical (insulin) drug chamber 103. The flexible plastic outer shell hinge 101 is situated above the ring-shaped magnet and the miniature motor controller 102, and the magnetic sensor 104 is positioned above the flexible plastic outer shell hinge 101.

As illustrated in FIG. 1A, the excitation/contraction of the microneedle diagram shows that the cylindrical microneedle wireless capsule 100 of the present invention, which can be controlled by an “externally applied magnetic field”, and then, microneedles 110 that can protrude laterally in the stomach. These microneedles are referred to as the 25G Carr-Locke needle (Steris Carr-Locke Injection Needle 25 gauge, 2.5 mm×230 cm).

The present invention is a cylindrical microneedle wireless capsule suitable for oral biopharmaceutical delivery. The schematic of the action after ingestion is depicted in FIG. 1B, showing the ingestion of the cylindrical microneedle wireless capsule. As shown in FIG. 1C, the capsule reaches the measurement position. FIG. 1D illustrates the release of biopharmaceuticals from the cylindrical microneedle wireless capsule.

As shown in FIG. 1E, the operation method of the cylindrical microneedle wireless capsule system of the present invention, the steps for controlling the release of biopharmaceuticals by the cylindrical microneedle wireless capsule are as follows:

The operation method of the cylindrical microneedle wireless capsule system of the present invention, as shown in FIG. 1E, step 10 is the “Start” step.

The operation method of the cylindrical microneedle wireless capsule system of the present invention, as shown in FIG. 1E, step 11 is the “Move to the desired injection position” step.

The operation method of the cylindrical microneedle wireless capsule system of the present invention, as shown in FIG. 1E, step 12 is the “Determine if an electrical stimulation position” step.

The operation method of the cylindrical microneedle wireless capsule system of the present invention, as shown in FIG. 1E, step 13 is the “Wait for the command from the remote end” step.

The operation method of the cylindrical microneedle wireless capsule system of the present invention, as shown in FIG. 1E, step 14 is the “Generate the first electrical pulse to set the timer for counting” step.

The operation method of the cylindrical microneedle wireless capsule system of the present invention, as shown in FIG. 1E, step 15 is the “Generate the second electrical pulse to set the timer for counting” step.

The operation method of the cylindrical microneedle wireless capsule system of the present invention, as shown in FIG. 1E, step 16 is the “Determine whether to continue stimulation or not” step.

The operation method of the cylindrical microneedle wireless capsule system of the present invention, as shown in FIG. 1E, step 17 is the “Reduce the timer count of the first electrical pulse and the second electrical pulse” step.

The operation method of the cylindrical microneedle wireless capsule system of the present invention, as shown in FIG. 1E, step 18 is the “Determine if the timer count of the electrical pulse equals to a zero” step.

The operation method of the cylindrical microneedle wireless capsule system of the present invention, as shown in FIG. 1E, step 19 is the “End” step.

The operation method of the cylindrical microneedle wireless capsule system of the present invention, as shown in FIG. 1E, steps 14 to 19 are the steps to “Execute the command received from the remote end”.

The present invention is a cylindrical microneedle wireless capsule 100, which owns a movable annular magnet and a miniature motor controller 102, the present invention can be displaced under a strong magnetic field condition. When the cylindrical microneedle wireless capsule 100 moves to a lesion in the body, the computer receiver will send a positioning signal to stop the present invention. At the target location, an external strong magnetic field is added to extend the microneedle 110. Through the rotation of the magnet, the flexible plastic shell hinge 101 is opened, and the microneedle 110 is connected to the injection point.

The present invention is a cylindrical microneedle wireless capsule 100. Inside the cylindrical microneedle wireless capsule 100, an Intel FPGA1SX280HU2F50E1VG chip can be installed, in order to generate a set of small magnetic fields, amplified by a Programmable Gain Amplifier (PGA). Moreover, multiple channels of the magnetic sensor 104 must be sampled simultaneously to obtain an electrical signal in the same magnetic field signal. For effectively supporting the battery power of the cylindrical microneedle wireless capsule 100, a Power Management Unit (PMU) can be provided to save power. The operating steps of the cylindrical microneedle wireless capsule 100 are as follows:

Step (A): The cylindrical microneedle wireless capsule 100 is affected by an upward magnetic force, causing the inner ring magnet to rotate around the bearing, serving as a magnetic marker to locate the cylindrical microneedle wireless capsule 100.

Step (B): When the inner ring magnet rotates, the magnetic poles of the two annular magnets and the miniature motor controller 102 are at one end, generating a repulsive force by using the HMC1023 magnetic sensor 104, and, pushing the two annular magnets and the miniature motor controller 102 attached to the puncture bearing to the top.

Step (C): On the top side of the cylindrical microneedle wireless capsule 100, three HMC1023 magnetic sensors are used to generate a set of repulsive forces for ejecting the microneedles.

Step (D): When the external magnetic field disappears, the attraction between the two annular magnets, and the miniature motor controller 102 causes the two annular magnets and the miniature motor controller 102 to rotate. When the two annular magnets and the miniature motor controller 102 start to rotate, the attractive force makes the two annular magnets and the miniature motor controller 102 close. Once the closure of the two annular magnets and the miniature motor controller 102 is completely closed, the objective of stimulating insulin ejection can be achieved.

The advantages of the cylindrical microneedle wireless capsule of the present invention include the following:

Advantage (A): the present invention possesses high compatibility and can be remotely controlled to manage the puncture needle (microneedle).

Advantage (B): the present invention owns an easily swallowable structure for drug release which uses a magnetic capsule endoscope.

Advantage (C): the magnetic capsule can use an externally controlled permanent magnet, the magnetic needle-like capsule is fixed on the tissue wall by magnetic force and rotates through the magnetic moment.

Advantage (D): the present invention provides linear movement behaviour, and the direction of the stable movement of the magnetic capsule is controllable, especially during non-linear movement processes, the present invention can achieve a stable state. In other words, the invention owns a miniature vibrating motor (RSR7/10-T010). This miniature vibrating motor can drive the rotor by the magnetic force of the fixed stator to rotate. Moreover, by changing the direction of the current in the coil, the direction of the magnetic force can be changed. This serves as an effective payload for encapsulating the drug and acts as the motive force for transdermal delivery.

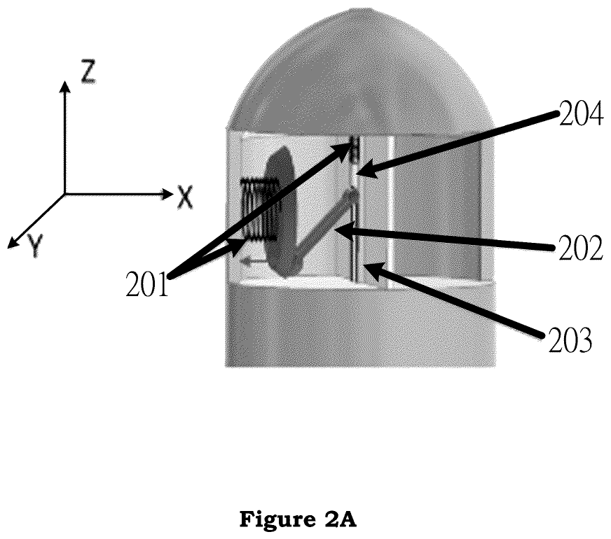

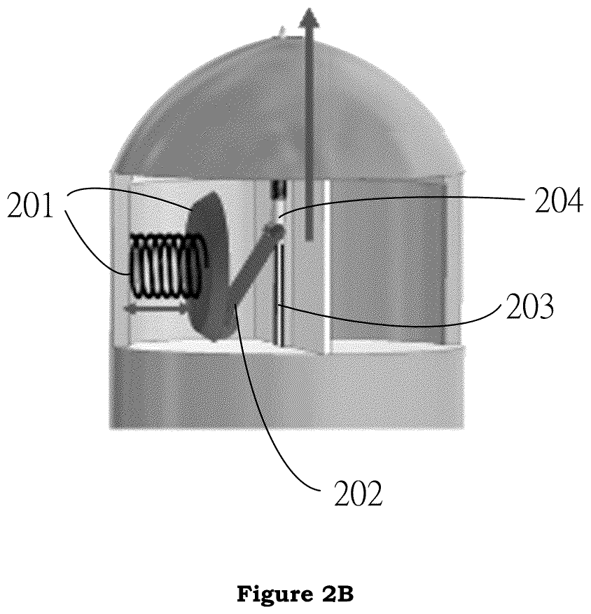

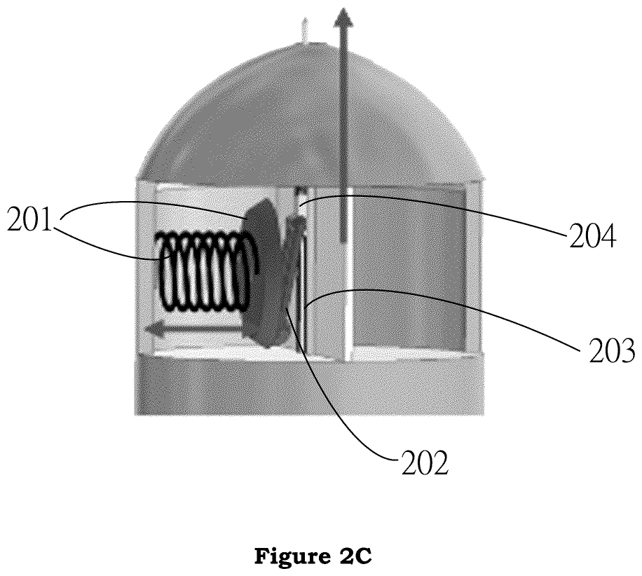

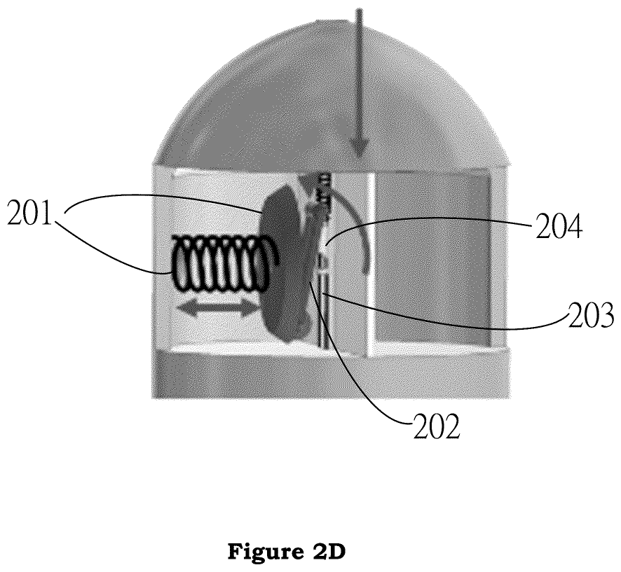

FIGS. 2A, 2B, 2C, and 2D display schematic diagrams of the microneedle action of the cylindrical microneedle wireless capsule 100 of the present invention. This includes FIG. 2A representing the extension/contraction of the puncture needle, FIG. 2B showing the swallowing of the capsule, FIG. 2C depicting the capsule reaching the measurement location, and FIG. 2D illustrating the drug release.

Referring to the abovementioned inventions, FIG. 2A represents the extension/contraction of the puncture needle, which consists of the spring component 201, connector 202, microneedle 203, and sliding part 204. The spring component 201 is connected to the connector 202, which in turn connects to the microneedle 203. The microneedle 203 is still located in the sliding part 204.

In FIG. 2B, which represents the swallowing of the capsule, when the spring component 201 springs forward, the connector 202 is pushed forward. Since the connector 202 is connected to the microneedle 203, the microneedle 203 moves in the sliding part 204, causing the microneedle 203 to extend outward.

FIG. 2C shows the capsule reaching the measurement location. At this point, the microneedle 203 will still continue to move forward within the sliding part 204, ensuring to keep extending outward.

In FIG. 2D, which illustrates the drug release, when the spring component 201 contracts, due to the connection to the connector 202, the connector 202 moves backward. This causes the microneedle 203 to move back in the sliding part 204, and then, the microneedle 203 retract.

FIG. 3 displays the control component 30 of the cylindrical microneedle wireless capsule of the present invention, which includes an application-specific integrated circuit (ASIC) 31, a Field Programmable Gate Array (FPGA) controller 32, a wireless antenna module (transmitter, STM32) 33, a power LED 34, and a battery 35. The ASIC 31 is electrically connected to the FPGA controller 32, which in turn is connected to the wireless antenna module 33. The battery 35 is electrically connected to the ASIC 31, then, the battery 35 is electrically connected to the FPGA controller 32, next, the battery 35 is electrically connected to the wireless antenna module 33, further, the battery 35 is electrically connected to the power LED 34.

In FIG. 3, the control component 30 of the cylindrical microneedle wireless capsule includes a dedicated ASIC 31. This ASIC contains a power decoder 301, an injection control structure array (32×32 SPAD Pixel Array) 302, a high voltage charge pump 303, 16-bit counters (pulse counting circuit) 304, a row decoder 305, and a data serializer 306. The power decoder 301 is electrically connected to the injection control structure array 302. The array 302 is electrically connected to the high voltage charge pump 303, and then, the array 302 is electrically connected to the 16-bit counters 304. The 16-bit counters 304 is electrically connected to the row decoder 305, and then, the row decoder 305 is electrically connected to the data serializer 306.

As demonstrated in FIG. 3 for the control component 30 of the cylindrical microneedle wireless capsule, the operation can adjust the pulse number released to control the dosage of a bio-pharmaceutical (e.g., insulin). The dosage resolution (DR) of pulse release, parameter design of the release module, are all related to the choice of micro-motor. The optimal motor step angle for the micro-motor is 5.6°, and the best motor reduction ratio is 1/64. The dosage resolution signifies the precision in controlling the release dosage of the bio-pharmaceutical (insulin).

As shown in FIG. 3 of the cylindrical microneedle wireless capsule's control component 30, when the FPGA controller 32 receives the drug release excitation signal via the wireless antenna module (STM32) 33, the capsule employs the ASIC 31's 0.35 μm high-voltage mixed-signal. Through an ultra-miniature vibration motor and the AD7745 capacitance-to-digital converter, acting as an independent capacitor with adjusted external pulse amplitude. At this point, the biopharmaceutical (insulin) microneedle immediately executes the drug release command. Upon completion, feedback is sent to the control component 30 of the capsule. When the FPGA controller 32 receives the microneedle's retraction signal, beginning the release process (approximately 12,500 pulses). A small amount of the drug might slightly overflow during the downward movement. The invention uses a new flexible silver oxide button battery from the Molex brand. The casing is made of high magnetic permeability Co—Ni—Fe alloy, which powers the capsule. Each battery measures about 7.7×2 mm, with the alloy casing being 0.2 mm thick.

This invention is a cylindrical microneedle wireless capsule suitable for oral biopharmaceuticals. The external diameter of microneedle is over 500 μm, enhancing the likelihood of successful perforation. The human stratum corneum (about 10 to 20 μm) can be penetrated up without causing cracks or bends, releasing biopharmaceuticals (including insulin) at predetermined areas and times. This functionality of capsule aids oral delivery and serves as an auxiliary platform for a plurality of biopharmaceuticals currently limited to injection-only treatment.

The cylindrical microneedle wireless capsule of the present invention can achieve controlled drug solubility curves, exhibiting excellent drug release kinetics. The appropriate dissolution rate of capsule microneedle, combined with skin permeation and clamping, enhances biocompatibility within skin tissues. Lastly, through 3D printing method, forming a helical structure made of plastic.

The development of this cylindrical microneedle wireless capsule is inspired by nature, drawing from elements like the spines of porcupines, snake fangs, tentacles of clams and octopuses, internal parasites, and the serrated microstructure of mantis forelegs. This invention employs protruding nano-microneedles that perform rapid separative motions in the skin, with the ultrasonic capsule capable of rotational movements on a supporting base, exhibiting clamp-like features during skin insertion. Controlled by the rotational magnetic field of a three-axis “Helmholtz” coil system, the invention utilizes soluble microneedles, gold-plated surface adhesives, and various microdevices to uniquely deliver medications, assisting in the delivery of traditional injectable biopharmaceuticals like insulin.

The cylindrical microneedle wireless capsule of this invention introduces a novel drug administration method. The microneedle array with microchannels can promote the penetration of therapeutic drug molecules into the epidermis and dermis of human, facilitating subsequent localized or systemic effects. Not only assists in achieving acceptable bioavailability comparable to standard injections, but also allows the drug to safely pass through the gastrointestinal tract. Moreover, the invention integrates swallowable capsule technology with Intel® FPGA and microneedle capsule technology, helping deliver biopharmaceuticals to parts of the body via the gastrointestinal tract.

It is understood that various other modifications will be apparent to and can be readily made by those skilled in the art without departing from the scope and spirit of the invention. Accordingly, it is not intended that the scope of the claims appended hereto be limited to the description as set forth herein, but rather that the claims be construed as encompassing all the features of patentable novelty that reside in the present invention, including all features that would be treated as equivalents thereof by those skilled in the art to which the invention pertains.

What is claimed is:

1. A cylindrical microneedle wireless capsule system, comprising:
a cylindrical microneedle wireless capsule; and
a control component of said cylindrical microneedle wireless capsule, wherein said control component of said cylindrical microneedle wireless capsule is electrically connected to control said cylindrical microneedle wireless capsule.

2. The cylindrical microneedle wireless capsule system according to claim 1, wherein said cylindrical microneedle wireless capsule comprising:
a flexible plastic outer shell hinge;
a ring-shaped magnet and miniature motor controller;
a biopharmaceutical drug chamber;
a magnetic sensor; and
a microneedle, wherein the microneedle being installed inside said biopharmaceutical drug chamber, said ring magnet and miniature motor controller are located said biopharmaceutical drug chamber, said flexible plastic outer shell hinge being located said ring-shaped magnet and miniature motor controller, and said magnetic sensor is located above said flexible plastic outer shell hinge.

3. The cylindrical microneedle wireless capsule system according to claim 2, wherein said ring magnet and miniature motor controller comprises two ring magnets.

4. The cylindrical microneedle wireless capsule system according to claim 2, wherein said microneedle comprises one outer diameter size of said microneedle exceeding 500 micrometers.

5. The cylindrical microneedle wireless capsule system according to claim 1, wherein said control component of said cylindrical microneedle wireless capsule comprising:
an application-specific integrated circuit;
a Field-Programmable Gate Array Controller;
a wireless antenna module;
a power LED; and
a battery, wherein the integrated circuit being electrically connected to said Field-Programmable Gate Array Controller, said Field-Programmable Gate Array Controller being electrically connected to said wireless antenna module, said battery being electrically connected to said integrated circuit, said battery being electrically connected to said Field-Programmable Gate Array Controller, said battery being electrically connected to said wireless antenna module, and said battery is electrically connected to said power LED.

6. The cylindrical microneedle wireless capsule system according to claim 5, wherein said application-specific integrated circuit of said control component of said cylindrical microneedle wireless capsule comprising:
a power decoder;
an injection control structure array;
a high voltage charge pump;
a pulse counting circuit;
a row decoder; and
a data serializer.

7. The cylindrical microneedle wireless capsule system according to claim 5, wherein said battery of said control component of said cylindrical microneedle wireless capsule comprises one flexible silver oxide button cell battery.

8. A method of operating a cylindrical microneedle wireless capsule system, comprising:
initiating;
moving to a desired injection position;
determining an electrical stimulation position;
waiting for a remote end to send or receive instructions;
generating a first electrical pulse to set a timer for counting;
generating a second electrical pulse to set said timer for counting;
determining whether to continue stimulation or not;
reducing an electrical pulse timer count;
determining if said electrical pulse timer count equals to a zero; and
ending.
